SEQUENCE LISTING (1 sequence)

SEQ ID NO: 1 - RNA, 1296 nt, Marinomonas phage
uuaaaaauguaaguuugcuucauuuugaccccucuugaguuuuaugcucaguaguuuuugcguuaauugcuuggugcuagcguggcgcgcauggcccagccagcuuugaauauuuugcuguaugucgauuagaucgaccucgccagcggcguauuucuuuugaaauuuacgcagcuugguuuuaauucuuuucacgcugcauuugcguaacaaacgaugcguugaguaaauccgauagccgagaaaauccaacgcccucccguuguguuuugcaaucgggaauacuugcguuuugcuguuuguuugcagucucaauugugaccacagaaaccgcucgaucuguucgcgccacucaugcaacugcgcuuugucgugaugaaugacugugaaaucauccauauaacgcauguaauuuggcgcuuuuaauguguguuuuacgaaccaauccaacucaugcaaauacacauuggcaaaaacuuggcuagucaaauugcccagcgguaugccgacgccugccgcaucgcuugggcuguugucgauaauguaaaacaacagugcuuucgugcguuggcaccuuaauuuuuuaucuaauaucgauuucaauauacugugaucgaugcucgaaaaauacuugcuaauguccgcuuuuagcgcgaacgcuuugccguguuuacgcuccacuugcuguauaaacgauugcgcccgauccgcgccuuuaugcguaccuuuguuuuugcggcacgcauaggaaucauagauauauuggcgaucaaacagcgguucgaugauguuauaaauugcccuaugcacaacucuaucccgaaaaugcggugcgcuaauuagccgacguuuuggcucaaacacauaaaaaugaugauagagcgacauuugauacaucugccaaacuaacucguuuuguaugcugaucacguuuucuucgagauuguuaaaaaacgacaauaccgcuguguuuguuguuuuaccgagccgacacugauaugccgcauucaguauguuuucaaacugauaaaucuguucguauaaacacucgcucgacgcaucgagcgagguaauucguguuucggcaauagccgaggaugcugcauccuuuucaugacugcacugacagcacgccuuggcguugcuguuucuggcauuaucaagagcggggcggaacccgauguucguguucgaauucgaacgagcauuguucagauugagcgcgcccaacccggcauucgagccguuguuccaauugccaccgcguagcgggaaucgguuccgcuuauucaucau

(19) United States
(12) Patent Application Publication
(10) Pub. No.: US 2021/0284977 A1
(43) Pub. Date: Sep. 16, 2021

(54) REVERSE TRANSCRIPTASE SEQUENCE OF MARINE BACTERIOPHAGE AND USE THEREOF

(71) Applicant: OCEAN UNIVERSITY OF CHINA, Qingdao (CN)

(72) Inventors: Min WANG, Qingdao (CN); Yundan LIU, Qingdao (CN); Yantao LIANG, Qingdao (CN); Hongbing SHAO, Qingdao (CN); Huifang LI, Qingdao (CN); Zhaoyang LIU, Qingdao (CN)

(21) Appl. No.: 17/052,508

(22) PCT Filed: Mar. 16, 2019
(86) PCT No.: PCT/CN2019/078408

§ 371 Date: Nov. 2, 2020

(30) Foreign Application Priority Data
Dec. 4, 2018 (CN) 201811473126.5

(51) Int. Cl. C12N 9/12; C12N 15/74

(52) U.S. Cl. CPC C12N 9/1276; C12N 15/74; C12Y 207/07049

(57) ABSTRACT

Disclosed is a reverse transcriptase sequence of marine bacteriophage and the use thereof in molecular biology.

### CROSS REFERENCE TO RELATED APPLICATION

This application claims the priority of Chinese Patent Application No. 201811473126.5, entitled “Reverse transcriptase sequence of marine bacteriophage and use thereof” filed with China National Intellectual Property Administration on Dec. 4, 2018, which is incorporated herein by reference in its entirety.

### TECHNICAL FIELD

The disclosure belongs to the technical field of microbial engineering, and particularly relates to a reverse transcriptase sequence of a marine bacteriophage and use thereof.

### BACKGROUND ART

Reverse transcription is a process in which RNA is used as a template to synthesize cDNA through reverse transcriptase. Reverse transcription is a special way of DNA biosynthesis, which is catalyzed by reverse transcriptase. American scientists H. M. Temin and D. Baltimore discovered reverse transcriptase in 1970, and therefore won the Nobel Prize in Physiology or Medicine in 1975. The discovery of reverse transcription has great impetus in promoting the development of molecular biology and genetic engineering technology. Reverse transcription is an indispensable tool in molecular cell biology experiments such as construction and expression of target genes of eukaryotic or prokaryotic, construction of cDNA libraries, and detection of gene sequences. Reverse transcription and Taq polymerase are the basic tools of modern biotechnology.

Bacteriophage is a type of virus that is ubiquitous and large in number in the ocean. Basic researches on the isolation, identification, biological characteristics and genome of marine bacteriophage play a vital role in understanding mortality of marine bacterial, biological carbon cycle, microbial community structure and horizontal gene transfer of marine microorganisms. However, due to the particularity of the bacteriophage structure and technical limitations, marine bacteriophage research started later than other organisms. In the early 1990s, people began to have a preliminary understanding of bacteriophages, and their ecological significance was gradually recognized. However, our current understanding of marine bacteriophages is merely a small part of it, especially in terms of the isolation of marine bacteriophages. Only nearly 2000 strains of tailed phage have been isolated. Most of the whole genome information obtained is unknown sequences, which greatly limits the in-depth research and use of marine bacteriophages. Therefore, in-depth understanding of marine bacteriophages has become a hot issue in marine ecology that needs to be addressed urgently.

In addition, the known reverse transcriptases are mainly derived from RNA viruses of animals, and no reverse transcriptase derived from marine bacteriophage has been found.

### SUMMARY OF THE INVENTION

The purpose of the disclosure is to provide a transcriptase sequence of marine bacteriophage to overcome the deficiency of the existing reverse transcriptases, to provide a new research tool for molecular biology, and thus promote its research and application in molecular biology, biochemistry and virology.

The identification of bacteria and bacteriophage typing are highly specific. A bacteriophage can only lyse one or similar bacterium, so it can be used for identification and typing of bacteria. At present, bacteriophage has been used to divide Staphylococcus aureus into four groups. This method of typing with bacteriophage is very helpful in tracing and analyzing the source of infection of these bacterial infections in epidemiological investigations.

Detecting bacteria in samples should use the bacteriophage titer growth test to check the corresponding bacteria in the sample. If a certain bacteriophage is detected in the sample, it often indicates the presence of the corresponding bacterium.

To achieve the above purpose, the specific technical schemes adopted by the disclosure are:

A reverse transcriptase sequence of marine bacteriophage, wherein the gene sequence of the reverse transcriptase is as follows:

UUAAAAAUGUAAGUUUGCUUCAUUUUGACCCCUCUUGAGUUUUAUGCUCA
GUAGUUUUUGCGUUAAUUGCUUGGUGCUAGCGUGGCGCGCAUGGCCCAGC
CAGCUUUGAAUAUUUUGCUGUAUGUCGAUUAGAUCGACCUCGCCAGCGGC
GUAUUUCUUUUGAAAUUUACGCAGCUUGGUUUUAAUUCUUUUCACGCUGC
AUUUGCGUAACAAACGAUGCGUUGAGUAAAUCCGAUAGCCGAGAAAAUCC
AACGCCCUCCCGUUGUGUUUUGCAAUCGGGAAUACUUGCGUUUUGCUGUU
UGUUUGCAGUCUCAAUUGUGACCACAGAAACCGCUCGAUCUGUUCGCGCC
ACUCAUGCAACUGCGCUUUGUCGUGAUGAAUGACUGUGAAAUCAUCCAUA
UAACGCAUGUAAUUUGGCGCUUUUAAUGUGUGUUUUACGAACCAAUCCAA
CUCAUGCAAAUACACAUUGGCAAAAACUUGGCUAGUCAAAUUGCCCAGCG
GUAUGCCGACGCCUGCCGCAUCGCUUGGGCUGUUGUCGAUAAUGUAAAAC
AACAGUGCUUUCGUGCGUUGGCACCUUAAUUUUUUAUCUAAUAUCGAUUU
CAAUAUACUGUGAUCGAUGCUCGAAAAAUACUUGCUAAUGUCCGCUUUUA
GCGCGAACGCUUUGCCGUGUUUACGCUCCACUUGCUGUAUAAACGAUUGC
GCCCGAUCCGCGCCUUUAUGCGUACCUUUGUUUUUGCGGCACGCAUAGGA
AUCAUAGAUAUAUUGGCGAUCAAACAGCGGUUCGAUGAUGUUAUAAAUUG
CCCUAUGCACAACUCUAUCCCGAAAAUGCGGUGCGCUAAUUAGCCGACGU
UUUGGCUCAAACACAUAAAAAUGAUGAUAGAGCGACAUUUGAUACAUCUG
CCAAACUAACUCGUUUUGUAUGCUGAUCACGUUUUCUUCGAGAUUGUUAA
AAAACGACAAUACCGCUGUGUUUGUUGUUUUACCGAGCCGACACUGAUAU
GCCGCAUUCAGUAUGUUUUCAAACUGAUAAAUCUGUUCGUAUAAACACUC
GCUCGACGCAUCGAGCGAGGUAAUUCGUGUUUCGGCAAUAGCCGAGGAUG
CUGCAUCCUUUUCAUGACUGCACUGACAGCACGCCUUGGCGUUGCUGUUU
CUGGCAUUAUCAAGAGCGGGGCGGAACCCGAUGUUCGUGUUCGAAUUCGA
ACGAGCAUUGUUCAGAUUGAGCGCGCCCAACCCGGCAUUCGAGCCGUUGU
UCCAAUUGCCACCGCGUAGCGGGAAUCGGUUCCGCUUAUUCAUCAU.

The specific screening and extraction process of the above reverse transcriptase is as follows:

(1) Preparation of Various Bacteria Solutions

The surface seawater was diluted gradually, then it was coated on the LB solid medium, and cultured; subsequently, the surface seawater was isolated and purified multiple times by the method of scraping line, and the various bacteria isolated was stored at 4° C. to reserve, and a variety of fresh bacteria solutions were obtained;

(2) Isolation and Purification of Marine Bacteriophages

The bacteriophage was isolated after the seawater being filtered with 0.22 membrane, the filtered water sample and the fresh bacteria solution obtained above were mixed at 1:1 (v/v), after infection of the bacteriophage, the bacteriophage was isolated by using the double-plate method, cultured at constant temperature, and the appearance of transparent plaques was observed;

If transparent plaques present, the plaques were extracted, they were placed in SM buffer, oscillated, filtered, the obtained bacteriophage sample was used to continue the double-plate experiment, and the results were observed, the process was repeated for 3 to 5 times, until the morphology and size of plaques are all similar, the isolated marine bacteriophage was thus obtained, and the corresponding fresh bacteria solution was the host bacteria solution;

(3) Identification of Host Bacteria

The genomic DNA of the host bacteria in the above host bacteria solution was extracted, the 16SrDNA was identified, and the host bacteria was identified as Marinomonas dokdonensis;

(4) Sequencing of Marine Bacteriophage

The marine bacteriophage was named as Marinomonas phage LX. Whole-genome sequencing was conducted. The reverse transcriptase was found at the ORF 4 site of its genome, which is a RNA-dependent DNA polymerase.

The discovery of the reverse transcriptase sequence shows that the bacteriophage needs to integrate its genes into the DNA sequence of the host bacteria through reverse transcription. In this process, there is a possibility of gene deletion, replacement, and even a large number of sequence rearrangements are likely to occur, leading to some functions of host bacteria changed, such as some toxic bacteria lose the ability to infect, and certain bacteria render with reduced or promoted rate of metabolism.

Bacteriophage is an important tool for molecular biology research. The number of bacteriophage genes is small. Some bacteriophages are genetically defective strains. Some bacteriophages can be easily controlled and identified by artificially induced variation and genetics, and can be used for gene transduction and conversion research. In recent years, bacteriophages have become the main gene carrier tool in genetic research.

The marine bacteriophage of Marinomonas phage LX and its reverse transcriptase may be used in molecular biology, especially in the preparation of medical clinical drugs.

Advantages and technical effects of the disclosure:

The discovery of reverse transcriptase sequence fills the gap in the research field of marine bacteriophage. It has great application prospects in the field of molecular biology. The bacteriophages may prompt new ideas for the treatment of certain clinical diseases. At the same time, bacteriophages can also be used to change the circulation of matter and energy in the ocean.

### BRIEF DESCRIPTION OF THE DRAWINGS

FIG. 1 is an electron micrograph of the marine bacteriophage of Marinomonas phage LX.

### DETAILED DESCRIPTION OF THE EMBODIMENTS

The disclosure will describe in further detail below with specific examples.

### Example 1

In this example, the nearshore surface seawater from Qingdao Lu Xun Park was used as the source of bacteriophage host bacteria for isolation. The surface seawater stored at 4° C. was gradually diluted with normal saline according to the actual concentration of the bacteria, and then it was coated on the LB solid medium. After sealing, it was placed at 28° C. and cultured overnight. Then it was isolated and purified multiple times by the method of scraping line, and the various bacteria isolated were stored at 4° C. to reserve. Part of the collected surface seawater was filtered with 0.22 μm filter membrane to remove larger particles, nanoplankton, bacteria and other impurities in the water to obtain a relatively pure virus water sample. 200 μL of the filtered water sample and 200 μL of fresh bacteria solution were mixed separately. After 20 minutes of infection of the bacteriophage, the bacteriophage was isolated by using the double-plate method. After culturing at a constant temperature of 28° C. for 24 hours, the appearance of transparent plaques was observed. The observed transparent plaques were poked with a pipette, and they were placed in SM buffer, oscillated and filtered, the obtained bacteriophage sample was used to continue the double-plate experiment, the results were observed, the above process was repeated for 3 to 5 times, until the morphology and size of plaques were all similar, and the isolated marine bacteriophage is thus obtained named as Marinomonas phage LX, and the corresponding strain was the host bacteria. The genomic DNA extraction of the obtained host bacteria was conducted, and 16S primer sequences 27F (5′-AGAXXXXX-3′) and 1492R (5′-AGAXXXXX-3′) were used for the primers of PCR amplification. After PCR amplification, electrophoresis was use for identification and sequencing. The results of 16S rDNA obtained by sequencing were matched with NCBI, and the host bacteria was identified as Marinomonas dokdonensis.

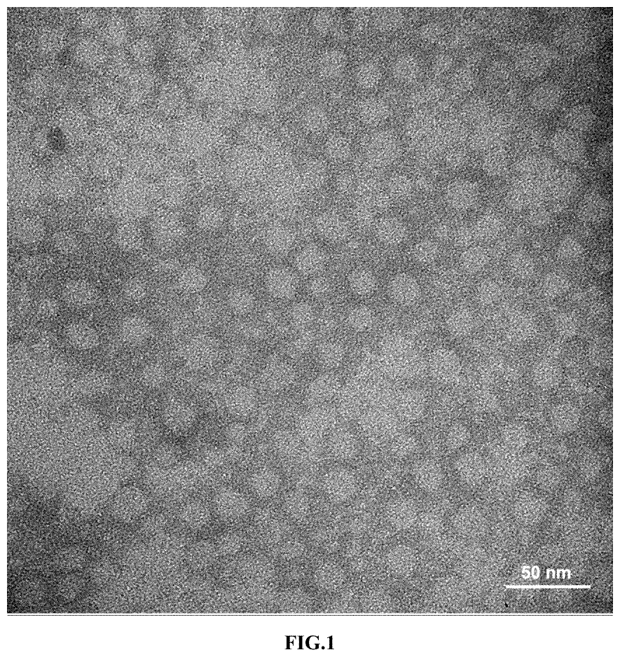

20 uL of stock solution of purified bacteriophage with a titer of 10¹⁰¹¹ pfu/mL was dropped on the copper net. After 15-minute natural sedment, 1 drop of 2% phosphotungstic acid (PTA) was added on the copper net, it was dyed for 10 minutes. The result was observed by electron microscope after drying. The bacteriophage was observed by electron microscope as tailless spherical phage with a size of 23 nm (FIG. 1).

Whole-genome sequencing was conducted on Marinomonas phage LX, and reverse transcriptase was found at the ORF 4 site of its genome, which is an RNA-dependent DNA polymerase.

The reverse transcriptase sequence is as follows:

UUAAAAAUGUAAGUUUGCUUCAUUUUGACCCCUCUUGAGUUUUAUGCUCA
GUAGUUUUUGCGUUAAUUGCUUGGUGCUAGCGUGGCGCGCAUGGCCCAGC
CAGCUUUGAAUAUUUUGCUGUAUGUCGAUUAGAUCGACCUCGCCAGCGGC
GUAUUUCUUUUGAAAUUUACGCAGCUUGGUUUUAAUUCUUUUCACGCUGC
AUUUGCGUAACAAACGAUGCGUUGAGUAAAUCCGAUAGCCGAGAAAAUCC
AACGCCCUCCCGUUGUGUUUUGCAAUCGGGAAUACUUGCGUUUUGCUGUU
UGUUUGCAGUCUCAAUUGUGACCACAGAAACCGCUCGAUCUGUUCGCGCC
ACUCAUGCAACUGCGCUUUGUCGUGAUGAAUGACUGUGAAAUCAUCCAUA
UAACGCAUGUAAUUUGGCGCUUUUAAUGUGUGUUUUACGAACCAAUCCAA
CUCAUGCAAAUACACAUUGGCAAAAACUUGGCUAGUCAAAUUGCCCAGCG
GUAUGCCGACGCCUGCCGCAUCGCUUGGGCUGUUGUCGAUAAUGUAAAAC
AACAGUGCUUUCGUGCGUUGGCACCUUAAUUUUUUAUCUAAUAUCGAUUU
CAAUAUACUGUGAUCGAUGCUCGAAAAAUACUUGCUAAUGUCCGCUUUUA
GCGCGAACGCUUUGCCGUGUUUACGCUCCACUUGCUGUAUAAACGAUUGC
GCCCGAUCCGCGCCUUUAUGCGUACCUUUGUUUUUGCGGCACGCAUAGGA
AUCAUAGAUAUAUUGGCGAUCAAACAGCGGUUCGAUGAUGUUAUAAAUUG
CCCUAUGCACAACUCUAUCCCGAAAAUGCGGUGCGCUAAUUAGCCGACGU
UUUGGCUCAAACACAUAAAAAUGAUGAUAGAGCGACAUUUGAUACAUCUG
CCAAACUAACUCGUUUUGUAUGCUGAUCACGUUUUCUUCGAGAUUGUUAA
AAAACGACAAUACCGCUGUGUUUGUUGUUUUACCGAGCCGACACUGAUAU
GCCGCAUUCAGUAUGUUUUCAAACUGAUAAAUCUGUUCGUAUAAACACUC
GCUCGACGCAUCGAGCGAGGUAAUUCGUGUUUCGGCAAUAGCCGAGGAUG
CUGCAUCCUUUUCAUGACUGCACUGACAGCACGCCUUGGCGUUGCUGUUU
CUGGCAUUAUCAAGAGCGGGGCGGAACCCGAUGUUCGUGUUCGAAUUCGA
ACGAGCAUUGUUCAGAUUGAGCGCGCCCAACCCGGCAUUCGAGCCGUUGU
UCCAAUUGCCACCGCGUAGCGGGAAUCGGUUCCGCUUAUUCAUCAU.

1. (canceled)

2. (canceled)

3. An isolated bacteriophage RNA sequence including SEQ. ID No. 1.

4. The isolated bacteriophage RNA sequence of claim 3, isolated according to a method comprising:
filtering a quantity of seawater to isolate a marine bacteriophage;
mixing the isolated marine bacteriophage with fresh seawater containing a host bacterium;
extracting genomic DNA of the host bacterium;
identifying the 16SrDNA of the host bacterium; and
determining the bacteriophage reverse transcriptase sequence within the host bacterium genome.

5. The isolated bacteriophage RNA sequence of claim 4, wherein the host bacterium is Marinomonas dokdonensis.

6. The isolated bacteriophage RNA sequence of claim 4, wherein the bacteriophage is Marinomonas phage LX.

7. A complementary DNA (cDNA) sequence synthesized from an RNA template using a reverse transcriptase sequence including SEQ. ID No. 1.

8. A method of altering a metabolic rate of a bacterium, the method comprising:
infecting the bacterium with a bacteriophage having a reverse transcriptase RNA sequence including SEQ. ID No. 1.
